(19) United States
(12) Patent Application Publication
(10) Pub. No.: US 2009/0005816 A1
(43) Pub. Date: Jan. 1, 2009

(54) Spinal rod, insertion device, and method of using

(76) Inventors: Andrew J. Denardo, Westfield, IN (US); Edward J. Morris, Bloomington, IN (US)

Correspondence Address: BAKER & DANIELS LLP, 300 NORTH MERIDIAN STREET, SUITE 2700, INDIANAPOLIS, IN, 46204, US

(21) Appl. No.: 11/821,897

(22) Filed: Jun. 26, 2007

(51) Int. Cl. A61B 17/70; A61B 17/58; A61B 19/00; A61N 1/30; A61F 5/00

(52) U.S. Cl. 606/246; 606/100; 606/96; 128/898; 604/19; 606/80; 606/254

(57) ABSTRACT

An apparatus and a method for fixing tissue.

### BACKGROUND AND SUMMARY

The present disclosure relates to an apparatus and a method for fusing bodily structures. More particularly, the present disclosure is directed to minimally invasive fusion of vertebral bodies.

Spinal fusion is often highly invasive and conducted via an open technique that exposes the vertebrae. Minimally invasive spine surgery techniques decrease the muscle retraction and disruption necessary to perform the same operation in comparison to the traditional open spinal fusion surgery.

Physicians have found that using a minimally invasive spine surgery system allows them to cause less soft tissue damage.

In one embodiment, a method of treating vertebrae of a spine is provided. The method including the steps of accessing a first vertebrae from the posterior via an access point; forming a first bore within the first vertebrae, the bore being at least partially aligned with the access point; forming a second bore within a second vertebrae that at least partially aligns with the bore in the first vertebrae; placing an adhesive within the second vertebrae; placing a support within the first and second bores; and placing adhesive within the first vertebrae.

In another embodiment, a kit including instruments for fusing vertebrae is provided. The kit including a first needle having a lumen and having a length suitable to extend from an extracorporeal position posterior of a vertebrae into the vertebrae; a second needle having a lumen and having an outer diameter sized to fit within the lumen of the first needle; a drill bit having an outer diameter sized to fit within the lumen of the second needle; and a support, the support being sized to fit within the lumen of the second needle.

### BRIEF DESCRIPTION OF THE DRAWINGS

FIG. 1 illustrates an introducer needle of the fusion system of the present disclosure partially disposed within a first vertebral body;

FIG. 2 illustrates a curved needle partially extending from the introducer needle of FIG. 1 within the first vertebral body;

FIG. 3 illustrates a drill bit partially extending from the curved needle of FIG. 2 within the first vertebral body;

FIG. 4 illustrates the curved needle of FIG. 2 and drill bit of FIG. 3 advanced into a second vertebral body;

FIG. 5 illustrates the curved needle and drill bit of FIG. 4 further advanced within the second vertebral body;

FIG. 6 illustrates a stabilizer secured within the first and second vertebral bodies; and

FIG. 7 illustrates an injector configured for delivery of bone cement for securing the stabilizer within the first and second vertebral bodies.

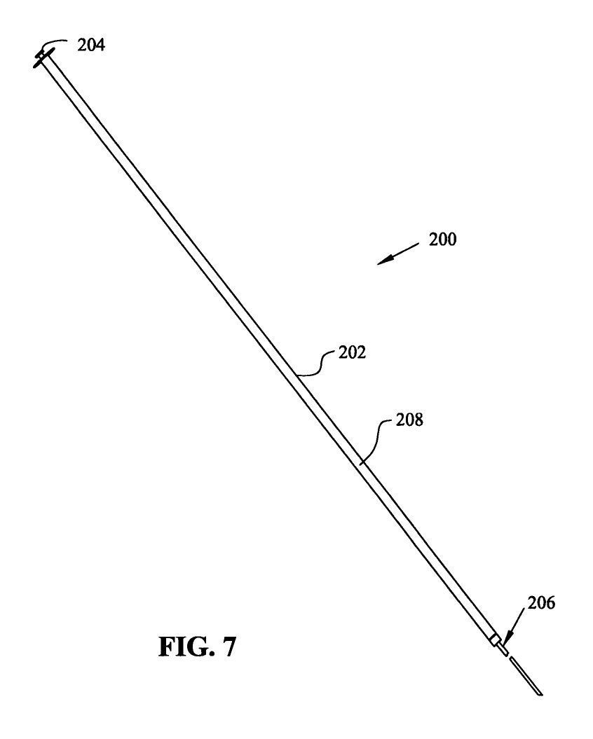

### DETAILED DESCRIPTION

The present disclosure is related to an apparatus and a method for fusing vertebrae. The apparatus and method of the present disclosure can be used to secure osteoporotic, injured, or otherwise unstable vertebrae. Additionally, the apparatus can be used in treatment for herniated or otherwise unstable intervertebral discs. Several factors are considered when designing and choosing a spinal fusion system. The easier the device is to use, the less experience necessary for a surgeon to effect a faster and less resource intensive surgery. Associated therewith is the learning curve associated with the device.

Operative time is a consideration. While related to ease of use, learning curve, and surgeon experience, operating room time is a resource and efficient use of that resource affects surgeon and medical facility profitability. Accordingly, operative time is a consideration in achieving industry acceptance. The size and number of incisions are considerations. On average, less tissue disruption, resection, and distraction are associated with improved results and decreased recovery time.

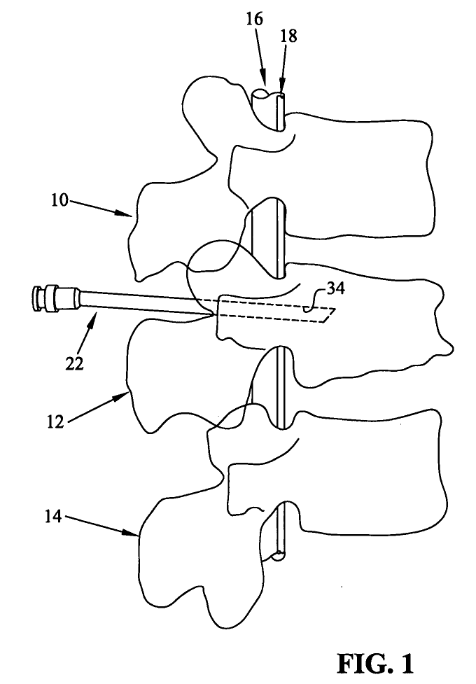

Referring now to the drawings, FIG. 1 is a right lateral view of a segment of a diseased spine. The segment includes three vertebral bodies/vertebrae 10, 12, 14, spinal cord 16, and epidural veins 18. Spinal cord 16 and epidural veins 18 run through the spinal canal of each vertebrae 10, 12, 14. Compressed vertebra 12 has a condition making it unstable and suitable for securing. Whereas the deterioration of compressed vertebra 12 causes/allows the spine to often assume a more curved orientation (the top would curve to the right in the orientation pictured or anteriorly), FIGS. 1-5 show the spine straightened in preparation for surgery.

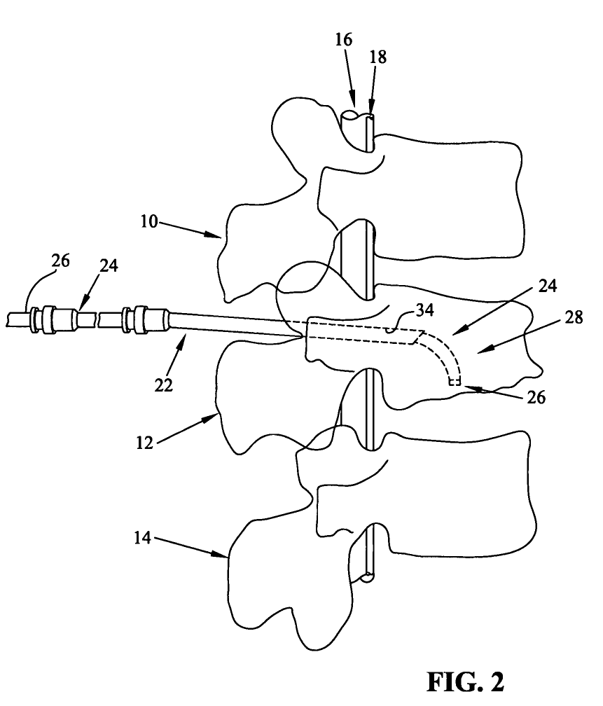

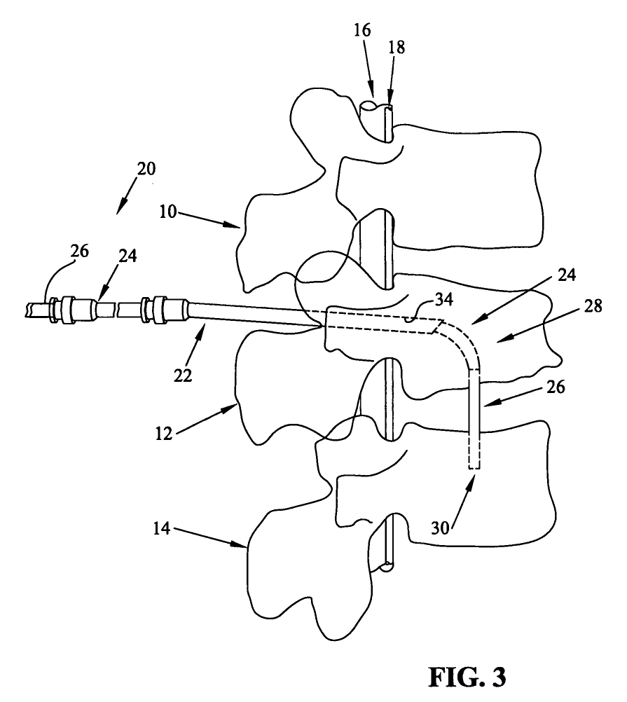

Device 20, shown assembled in FIGS. 3 & 4, includes introducer needle 22, curved needle 24, and drill bit 26. Introducer needle 22 is shown as an 11 gauge needle. However, needles 22 of different sizes may be used as needed for fixture rods 100 of different diameters. Curved needle 24 is flexible such that it may be straightened out to travel within a lumen of introducer needle 22. However, absent external force keeping curved needle 24 straight, curved needle 24 attempts to assume a rest state having ninety-degrees of curvature near distal end 28, as shown in FIGS. 2 & 3. Embodiments of curved needle 24 are envisioned that attempt to assume angles of curvature greater and less than ninety-degrees. Curved needle 24 has an outer diameter sized to be received within a lumen of introducer needle 22. Introducer needle 22 is of a stiffness such that distal end 28 of curved needle 24 is forced straight rather than curved needle 24 curving introducer needle 22.

Drill bit 26 is a flexible bit. Drill bit 26 has a tip suitable for boring into osseous tissue. Absent external force, drill bit 26, in opposition to curved needle 24, attempts to assume a straight rest state. The force of drill bit 26 that attempts to keep it straight is less than the force of curved needle 24 that attempts to keep it curved. Accordingly, the properties of curved needle 24 that cause it to be curved can overcome the properties of drill bit 26 that attempt to keep drill bit 26 straight. Thus, when drill bit 26 is placed within a lumen of curved needle 24, drill bit 26 curves according to the orientation of the curved needle 24.

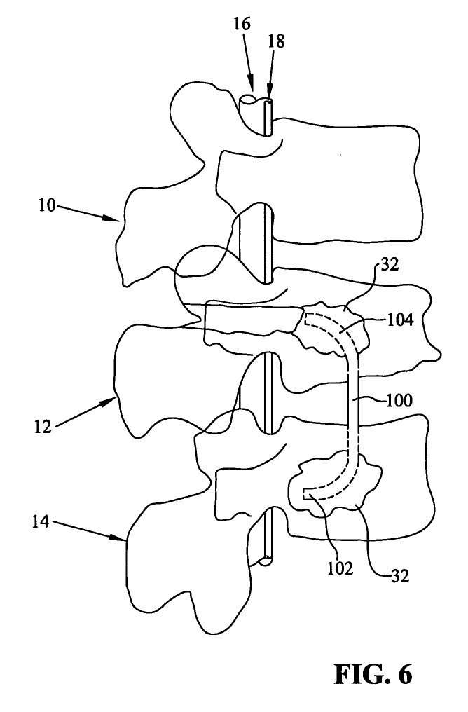

Fixture rod 100, FIG. 6, is shown as a nitinol rod, but other materials such as stainless steel and titanium are also envisioned. Having described rod 100 and the tools used in its placement, the method of rod placement will now be described. The process described below provides for a spinal fusion from a posterior approach. Whereas the placement of a single rod 100 is described, it should be appreciated that a patient may require multiple rods I 00 to be placed through repetition of the below described process.

FIG. 1 shows introducer needle 22 inserted in bore 34 in the posterior of compressed vertebra 12. Introducer needle 22 is inserted laterally of the spinal canal so as to not puncture spinal cord 16 or veins 18. Positioning of introducer needle 22 may be achieved via pressure from the surgeon, especially when vertebrae 12 is osteoporotic. Alternatively, drill bit 26 or a different drill bit is used to bore a hole within vertebrae 12 for introducer needle 22.

It should be appreciated that for all of introducer needle 22, curved needle 24, and drill bit 26, by coordinating with previously taken radiographs the locations of the respective distal ends may be ascertained by noting how far the piece (22, 24, 26) has been inserted into the bone or into the other piece (22, 24). For this purpose, the exterior of each piece 22, 24, 26 is provided with graduation marks that allow the surgeon to gauge how far the piece 22, 24, 26 has been inserted.

FIG. 2 shows curved needle 24 advanced within and out of introducer needle 22. Curved needle 24 assumes its curved orientation once it is free from the constraints of the introducer needle 22. Again, curved needle 24 can be advanced through pressure applied by the surgeon. Alternatively, drill bit 26 is slightly exposed, as shown in FIG. 2, and bores a path. By barely exposing drill bit 26, the path is dictated by curved needle 24. In such embodiments, curved needle 24 and drill bit 26 are advanced together to cut the arcuate path. Curved needle 24 is advanced until it achieves the ninety degree deflection shown in FIG. 2.

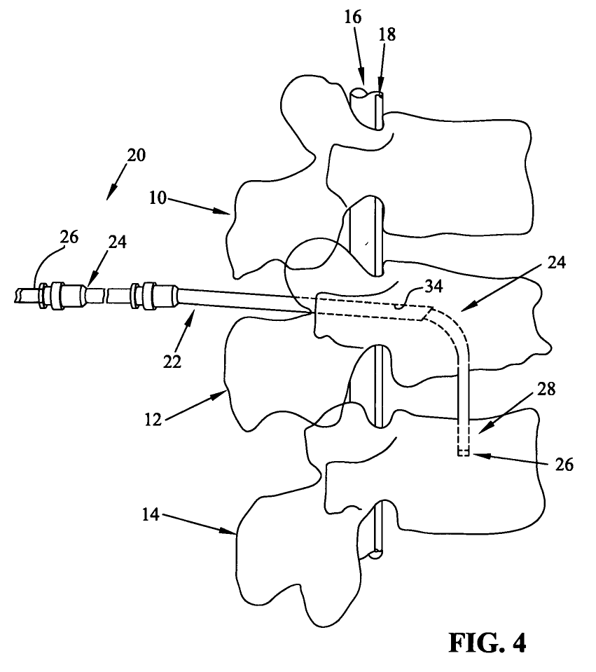

As shown in FIG. 3, drill bit 26 is then inserted further, while failing to advance curved needle 24, to cut a path out of compressed vertebrae 12, passing through intervertebral space and into lower vertebrae 14. As previously discussed, the bias of drill bit 26 to remain straight causes drill bit 26 to produce a straight cut when not restrained within curved needle 24, as shown in FIG. 3. Once drill bit 26 advances into lower vertebrae 14, the lateral position of drill bit 26 is then held by compressed vertebra 12 and lower vertebrae 14. With drill bit 26 so anchored curved needle 24 is advanced along drill bit 26. As previously noted, curved needle 24 is normally is able to impart a curved shape to drill bit 26. However, with distal end 30 of drill bit 26 restricted laterally by lower vertebrae 14, curved needle 24 advances linearly along straight drill bit 26 to the position shown in FIG. 4.

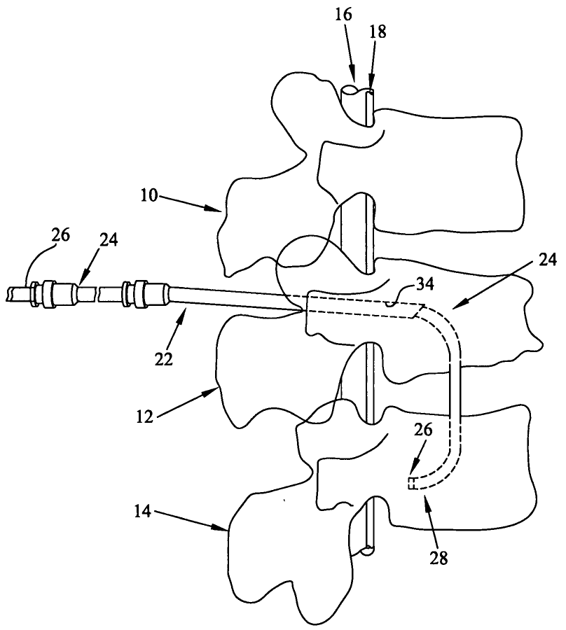

Then, curved needle 24 is advanced within lower vertebrae 14 either by pressure applied by the surgeon to needle 24 or via drill bit 26 that is slightly exposed from needle 24 and advanced together with needle 24 to cut an arcuate path. Curved needle 24 is advanced until it achieves the ninety degree deflection shown in FIG. 5. If desired, drill bit 26 can be extended further to posteriorly bore further (such a bore is shown in FIG. 6).

Subsequently, drill bit 26 is removed from curved needle 24 and the anatomy entirely. Injector 200, described below, is used to inject polymethyl methacrylate bone cement or another cement/adhesive 32 into lower vertebrae 14. Bone is a porous material. As such adhesive 32 spreads from the injection site into adjacent osseous tissue. Rod 100 is then inserted through curved needle 24 and introducer needle 22 such that distal end 102 thereof engages lower vertebrae 14 and adhesive 32 deposited there. Embodiments are also envisioned where rod 100 is placed simultaneously with the bone cement. A pusher, not shown, includes a distal end that engages rod 100 and is used to push rod 100 into place. Embodiments are envisioned where the pusher selectively engages rod 100 so that the pusher disengages from rod 100 only upon user instruction. Once rod 100 is properly placed, the pusher is removed. Curved needle 24 is then retracted to near proximal end 104 of rod 100. Alternatively, curved needle 24 may be retracted after adhesive 32 placement and before rod 100 placement.

After rod 100 placement, additional adhesive 32 is placed in compressed vertebrae 12 near proximal end 104 of rod 100 via injector 200. Again, adhesive 32 extends into the porous osseous tissue of compressed vertebrae 12. Additional cement/adhesive 32 is inserted to fill bore 34 of introducer needle 22. Finally, any remaining pieces of device 20 are removed. Standard closing up procedures are then performed for the surrounding soft tissue.

Whereas the path cut by device 20 for the location of rod 100 is shown to have straight and curved portions, embodiments are also envisioned where device 20 cuts a path for rod 100 such that rod 100 is totally arcuate when placed.

Injector 200 in the present example, takes the form of a syringe. Other embodiments of injector 200 are also envisioned that approximate other injectors such as caulk guns and the like. Injector 200 includes barrel 202, plunger 204, and needle 206. Needle 206 is flexible such that it can be easily bent to follow a non-linear path.

As previously noted, when injected, polymethyl methacrylate bone cement extends into the surrounding porous bone. The amount of travel into surrounding bone can be tailored to the desires of the application by choosing the viscosity of the cement. Polymethyl methacrylate having a higher viscosity is less likely to travel far within the osseous tissue when compared to lower viscous cement. The highly viscous cement has the advantage of being less likely to travel into unwanted places. However, higher viscosity cement is similarly less amenable to travel within injector 200. Higher viscosity cement requires greater pressure to move compared to the pressure necessary to move lower viscosity cement.

Injector 200 is configured to provide high pressure to allow the placement of highly viscous cement. Injector 200 is operated by a mechanical actuator (not pictured). Accordingly, it is not necessary that injector 200 be configured to fit in and be manipulated by a user's hand. In the shown example, barrel 202 has a substantially cylindrical load space 208 on the interior thereof. Load space 208 has a diameter of approximately 2.5 mm. Other embodiments have diameters of less than 2.5 mm. Load space 208 has a length of approximately 250 mm. Other embodiments have longer lengths up to three feet or more. The length to diameter ratio of load space 208 is approximately 100-1. Other envisioned embodiments have a length to diameter ratio of 100-1 or greater.

Once placed, rod 100 fuses vertebrae 12, 14. Rod 100 provides vertical support to counter compressive forces encountered by an upright posture. Additionally, rod 100 is somewhat flexible. Accordingly, at least some flexibility between vertebrae 12, 14 is usually maintained.

While certain embodiments of the present disclosure have been described in detail, those familiar with the art to which this disclosure relates will recognize various alternative designs and embodiments for practicing the disclosure as defined by the following claims.

What is claimed is:

1. A method of treating vertebrae of a spine, the method including the steps of:
accessing a first vertebrae from the posterior via an access point;
forming a first bore within the first vertebrae, the bore being at least partially aligned with the access point;
forming a second bore within a second vertebrae that at least partially aligns with the bore in the first vertebrae;
placing an adhesive within the second vertebrae;
placing a support within the first and second bores; and
placing adhesive within the first vertebrae.

2. The method of claim 1, further including the step of placing a first needle within the access point.

3. The method of claim 2, wherein each of the steps of forming the first bore and forming the second bore includes forming the bore by a drill bit that extends within the first needle.

4. The method of claim 2, wherein the step of placing a support includes extending the support through the first needle.

5. The method of claim 1, further including the step of placing a second needle within the first needle.

6. The method of claim 5, wherein the second needle is a flexible needle having a rest state wherein the second needle is curved.

7. The method of claim 5, further including the step of placing a drill bit within the second needle.

8. The method of claim 7, wherein the step of forming a first bore includes simultaneous advancing of the second needle and the drill bit within the first vertebrae.

9. The method of claim 7, wherein the step of forming a second bore includes advancement of the drill bit relative to the second needle.

10. The method of claim 7, wherein the step of forming a second bore includes simultaneous advancement of the second needle and the drill bit within the second vertebrae.

11. The method of claim 1, wherein the support is a rod.

12. The method of claim 1 1, wherein the rod includes straight portions and bent portions.

13. A kit including instruments for fusing vertebrae, the kit including:
a first needle having a lumen and having a length suitable to extend from an extracorporeal position posterior of a vertebrae into the vertebrae;
a second needle having a lumen and having an outer diameter sized to fit within the lumen of the first needle;
a drill bit having an outer diameter sized to fit within the lumen of the second needle; and
a support, the support being sized to fit within the lumen of the second needle.

14. The kit of claim 13, further including a pusher sized to fit within the lumen of the second handle and having a distal end configured to engage the support.

15. The kit of claim 13, further including an adhesive applicator.

16. The kit of claim 15, wherein the applicator includes a needle sized to fit within the lumen of the second needle.

17. The kit of claim 16, wherein the applicator includes a substantially cylindrical load space.

18. The kit of claim 17, wherein the load space has a diameter and a length, the ratio of the diameter to the length being at least 1:100.

19. The kit of claim 13, wherein the second needle is flexible and includes a portion, the portion having a rest state in which the needle is curved.

20. The kit of claim 19, wherein the portion having a curved rest state defines an arc of at least 30 degrees in the rest state.

21. The kit of claim 13, wherein the drill bit is flexible and has a rest state in which the drill bit is straight.
